(19) United States
(12) Patent Application Publication
(10) Pub. No.: US 2016/0228272 A1
(43) Pub. Date: Aug. 11, 2016

(54) STENT HAVING HIGH EXPANSION RATIO

(71) Applicant: Gil Vardi, Town & Country, MO (US)

(72) Inventor: Gil Vardi, Town & Country, MO (US)

(21) Appl. No.: 15/131,893

(22) Filed: Apr. 18, 2016

Related U.S. Application Data
(63) Continuation of application No. 11/809,955, filed on Jun. 4, 2007.

(51) Int. Cl. A61F 2/954; A61F 2/90; A61F 2/844; A61F 2/958

(52) U.S. Cl. CPC A61F 2/954; A61F 2/958; A61F 2/90; A61F 2/844; A61F 2250/0098; A61F 2230/0071; A61F 2230/0069

(57) ABSTRACT

A device for expanding a stent to treat a bifurcation within a body lumen has a catheter for delivering and positioning a stent to a bifurcation within a body lumen, the catheter having a balloon positioned adjacent to the stent, the balloon for being inflated to a predetermined configuration to expand the stent, the predetermined configuration having a first balloon portion, a central balloon portion, and a second balloon portion, the central balloon portion for being positioned adjacent to the bifurcation with the central balloon portion being inflated to expand the stent and to form an opening in the stent adjacent to the bifurcation to facilitate better flow into a branch associated with the bifurcation to prevent or limit a compromise or a closure of the branch, the central balloon portion being inflated to a larger diameter that the first balloon portion and the second balloon portion.

### CROSS REFERENCE TO RELATED APPLICATIONS

This application is a continuation of U.S. patent application Ser. No. 11/809,955 filed on Jun. 4, 2007.

### BACKGROUND

This disclosure relates generally to a stent having a high expansion ratio and, more particularly, to a stent including a frame that is movable between a collapsed configuration and an expanded configuration to treat a bifurcation.

Support structures that are collapsible but yet provide adequate strength and/or stiffness, particularly in a radial direction, are desirable when positioned within relatively small spaces. It is also desirable that such support structures are expandable to a deployed configuration greater than a collapsible, insertion configuration. Such support structures are useful in medical applications including, without limitation, implantation of luminal stents within a patient's vasculature.

Stents are generally utilized to support living tissues, such as a body lumen. For example, stents may be used to radially support a collapsing or occluded blood vessel or to treat an aneurysm within a blood vessel. Conventional stents typically form an elongated tube or wire frame that provides structural support for the vessel wall. Further, a tubular graft may be positioned about or within the stent to facilitate blood flow through the blood vessel while preventing undesirable blood flow into the aneurysm.

Many conventional stents are positioned in a collapsed, insertion configuration within a delivery system including a catheter to deliver the stent to a lesion site within the body lumen. With the stent properly positioned with respect to the lesion site, the stent is disengaged from the catheter and urged to expand to a deployed configuration.

### BRIEF SUMMARY

In another aspect, an expandable frame is provided. The expandable frame includes a first member including a plurality of substantially parallel first struts and a plurality of substantially parallel second struts with the frame in a collapsed configuration. Each second strut of the plurality of second struts is coupled between adjacent first struts of the plurality of first struts defining a plurality of open cells to facilitate expansion of the frame towards an expanded configuration. At least one second member is coupled to the first member. The at least one second member includes a plurality of third struts. Each third strut of the plurality of third struts is coupled to adjacent third struts at an acute angle with the frame in the collapsed configuration.

In another aspect, a stent is provided. The stent includes an expandable frame. The expandable frame includes a first member including a plurality of substantially parallel first struts and a plurality of substantially parallel second struts with the frame in a collapsed configuration. Each second strut of the plurality of second struts is coupled between adjacent first struts of the plurality of first struts defining a plurality of open cells to facilitate expansion of the frame towards an expanded configuration. At least one second member is coupled between a first point on the first member and a second point on the first member.

In another aspect, an expandable frame is provided. The expandable frame includes a first member including a plurality of first struts. Each first strut is coupled to adjacent first struts at an acute angle with the frame in a collapsed configuration to define a plurality of open cells to facilitate expansion of the frame towards an expanded configuration. At least one second member is coupled to the first member. The at least one second member includes a plurality of second struts. Each second strut is coupled to adjacent second struts at an acute angle with the frame in the collapsed configuration.

In another aspect, a stent is provided. The stent includes a first segment having a first expansion ratio, and a second segment having a second expansion ratio. The first segment and/or the second segment is radially expandable to form an opening at least partially defined within a body wall of the stent.

### BRIEF DESCRIPTION OF THE DRAWINGS

FIG. 1 is a schematic view of an exemplary stent having a high expansion ratio and including an expandable frame in a collapsed configuration;

FIG. 2 is a schematic view of a portion of the frame shown in FIG. 1 in the collapsed configuration;

FIG. 3 is a schematic view of a portion of the frame shown in FIG. 1 in an expanded configuration;

FIG. 4 is a schematic view of an alternative exemplary stent having a high expansion ratio and including an expandable frame having a helical configuration;

FIG. 5 is a schematic view of a portion of an alternative exemplary stent having a high expansion ratio and including an expandable frame in a collapsed configuration;

FIG. 6 is a schematic view of a portion of an alternative exemplary stent having a high expansion ratio and including an expandable frame in a collapsed configuration;

FIG. 7 is a schematic view of an alternative exemplary stent having a high expansion ratio;

FIGS. 8A-8C are schematic views of alternative exemplary stents having a high expansion ratio;

FIG. 9 is a system including a catheter and an inflatable balloon;

FIG. 10 is a system including a catheter and a series of inflatable balloon;

FIG. 11A is a schematic view of an exemplary catheter system including a stent positioned about a series of inflatable balloons and positioned with respect to a bifurcation point within a body lumen;

FIG. 11B is a schematic view of the catheter system shown in FIG. 11A with the balloons inflated to radially expand the respective stent segments;

FIG. 11C is a schematic view of the catheter system shown in FIG. 11A with radially expanded stent segments; and

FIG. 12 is a schematic view of an alternative exemplary catheter system including two stent segments positioned about a series of inflatable balloons and within a body lumen.

### DETAILED DESCRIPTION OF PREFERRED EMBODIMENTS

The present disclosure provides a device, such as a stent, having a high expansion ratio to facilitate properly positioning the device in a collapsed, insertion configuration within a space and/or expanding the device within the space to an expanded, deployed configuration. Although the present disclosure is described below in reference to its application in connection with a stent that is positioned within a body lumen to support and/or treat a portion of the body lumen, it should be apparent to those skilled in the art and guided by the teachings herein provided that the disclosure is likewise applicable for use with any suitable device or component, including any suitable device or component that is implanted within a patient's body.

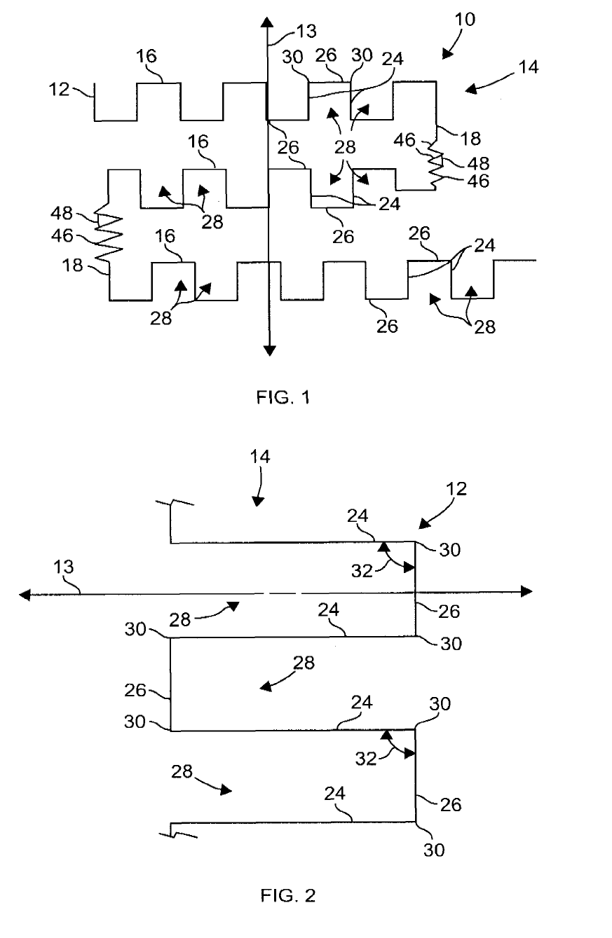

Referring to FIGS. 1-4, a stent 10 includes an expandable frame 12 or support structure that is expandable in a radial outward direction with respect to a longitudinal axis 13 defined by stent 10 and/or expandable along longitudinal axis 13. In a particular embodiment, frame 12 includes components that are extendible.

Frame 12 is fabricated of a biocompatible material including, without limitation, suitable metal materials, such as stainless steel, platinum, gold, titanium and nickel and/or composites or alloys thereof. In the exemplary embodiment, frame 12 is fabricated at least partially from a material having shape memory properties. Suitable materials include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, frame 12 is fabricated from a thermally treated metal alloy (TMA) including, without limitation, nickel titanium, beta titanium, copper nickel titanium and any combination thereof. In one embodiment, frame 12 is expandable using a balloon and/or another mechanism suitable for facilitating expanding frame 12. In an alternative embodiment, frame 12 is fabricated at least partially from a suitable polymeric material, such as a polyurethane material. It should be apparent to those skilled in the art and guided by the teachings herein provided that frame 12 may be made or fabricated using any suitable biocompatible material preferably, but not necessarily, having suitable shape memory properties.

Frame 12 includes at least one first member 14. In one embodiment as shown in FIG. 1, first member 14 includes a plurality of substantially parallel, expandable bands 16. At least one second member 18, as described in greater detail below, is coupled between adjacent bands 16 to affect the expandability and/or flexibility of frame 12. Bands 16 may define any suitable cross-sectional area including, without limitation, an elliptical cross-sectional area or a polygonal cross-sectional area.

FIG. 4 is a schematic view of an alternative exemplary frame 12. As shown in FIG. 4, frame 12 is formed in a helical configuration to define a suitable cross-sectional area. In a particular embodiment, frame 12 is formed of a continuous wire fabricated form a suitable material, as described above. In this alternative embodiment, first member 14 is formed in a circumferential direction to define a helical configuration having an elliptical cross-sectional area. Alternatively, first member 14 may define any suitable cross-sectional area including, without limitation, a suitable polygonal cross-sectional area. First member 14 includes a plurality of substantially parallel windings 20. In a particular embodiment, at least one second member 18 is coupled between adjacent windings 20.

First member 14 includes a plurality of first struts 24 (not shown in FIG. 4) and a plurality of second struts 26 (not shown in FIG. 4) coupled between adjacent first struts 24 to define a plurality of open cells 28 (not shown in FIG. 4) configured to facilitate expansion of first member 14. First member 14 defines any suitable number of open cells 28. First member 14 expands circumferentially as first struts 24 move with respect to adjacent second struts 26 such that frame 12 expands radially outward with respect to longitudinal axis 13. In one embodiment, each first strut 24 has a length different than a length of each second strut 26. Alternatively, each first strut 24 has a length substantially equal to a length of each second strut 26. In a particular embodiment, first struts 24 are substantially parallel with adjacent first struts 24. Further, second struts 26 are substantially parallel with adjacent second struts 26 and substantially perpendicular to first struts 24, with frame 12 in a collapsed or compressed configuration, such as shown in FIGS. 1 and 2.

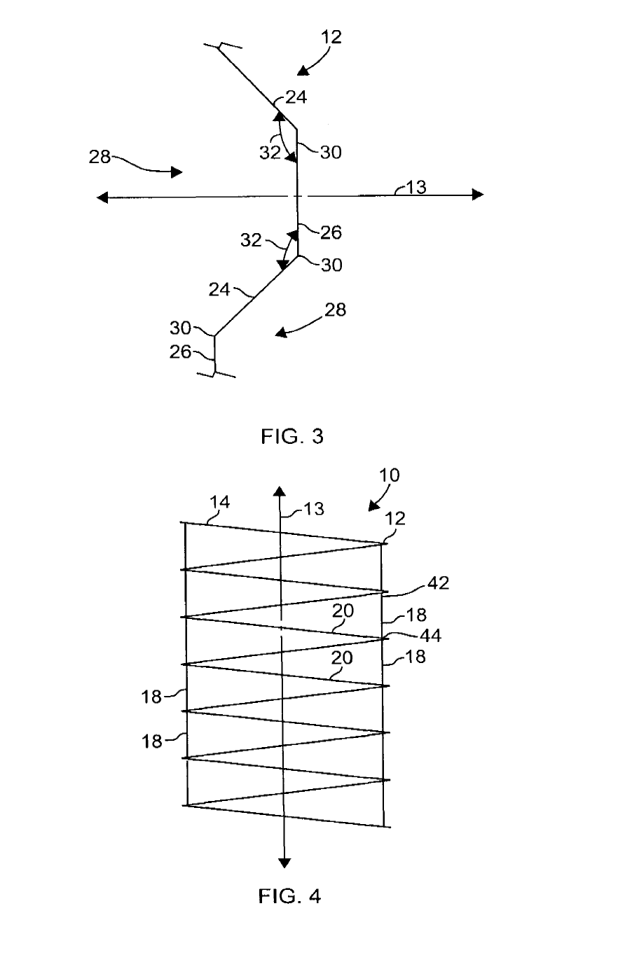

Second struts 26 are coupled between adjacent first struts 24 to facilitate expansion of frame 12 towards an expanded configuration, such as shown in FIG. 3. First strut 24 and adjacent second strut 26 are flexible to allow first strut 24 and/or second strut 26 to move, such as pivot or bend, with respect to a point 30 at which first strut 24 transitions into or is coupled to adjacent second strut 26 to facilitate expansion of frame 12. As frame 12 expands from the collapsed configuration, as shown in FIG. 2, towards the expanded configuration, as shown in FIG. 3, first strut 24 moves with respect to second strut 26 such that an angle 32 defined between first strut 24 and second strut 26 increases.

Referring further to FIGS. 1 and 4, frame 12 also includes at least one second member 18 coupled between two or more bands 16, as shown in FIG. 1, or between a first point 42 and a second point 44 on first member 14, as shown in FIG. 4, to affect expansion and/or flexibility of stent 10. Second member 18 includes a plurality of third struts 46 (not shown in FIG. 4) coupled to adjacent third struts 46 at an acute angle 48 with frame 12 in the collapsed configuration. As second member 18 expands, each third strut 46 moves with respect to adjacent third struts 46 such that angle 48 increases. In a particular embodiment, angle 48 may approach a right angle or an obtuse angle with frame 12 in the expanded configuration.

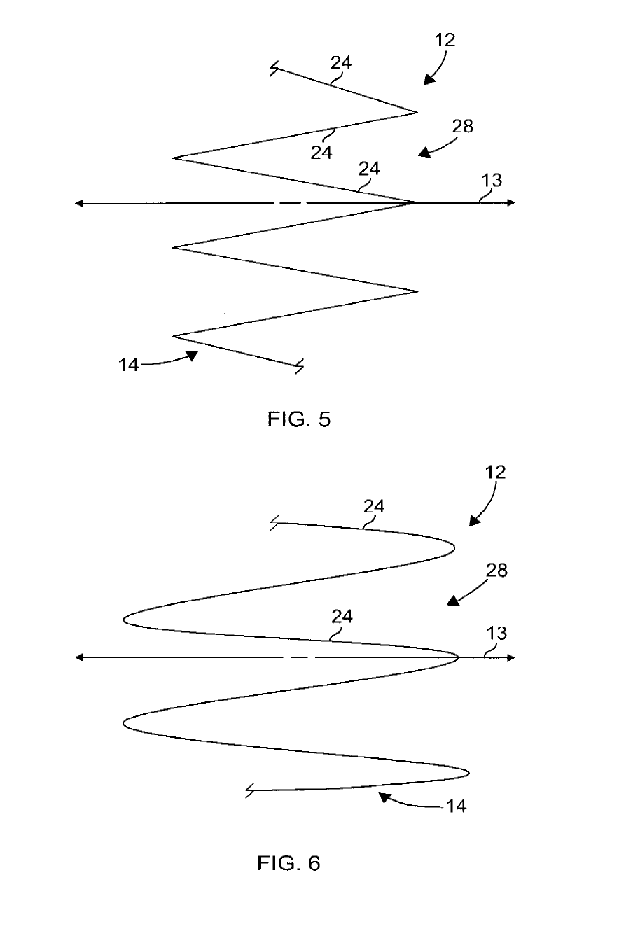

FIGS. 5 and 6 are schematic views of alternative exemplary frames 12. Referring to FIGS. 5 and 6, in alternative embodiments stent 10 includes expandable frame 12 or support structure that is expandable in a radial outward direction with respect to longitudinal axis 13 defined by stent 10 and/or expandable along longitudinal axis 13. Frame 12 includes at least one first member 14 including a plurality of first struts 24 coupled to or integrated with adjacent first struts 24 to define a plurality of open cells 28 configured to facilitate expansion of first member 14. First member 14 expands circumferentially as first struts 24 move with respect to adjacent first struts 24 such that frame 12 expands radially outward with respect to longitudinal axis 13. Adjacent first struts 24 may be configured in a zig-zag configuration, as shown in FIG. 5, or an arcuate configuration, such as a sinusoidal configuration as shown in FIG. 6.

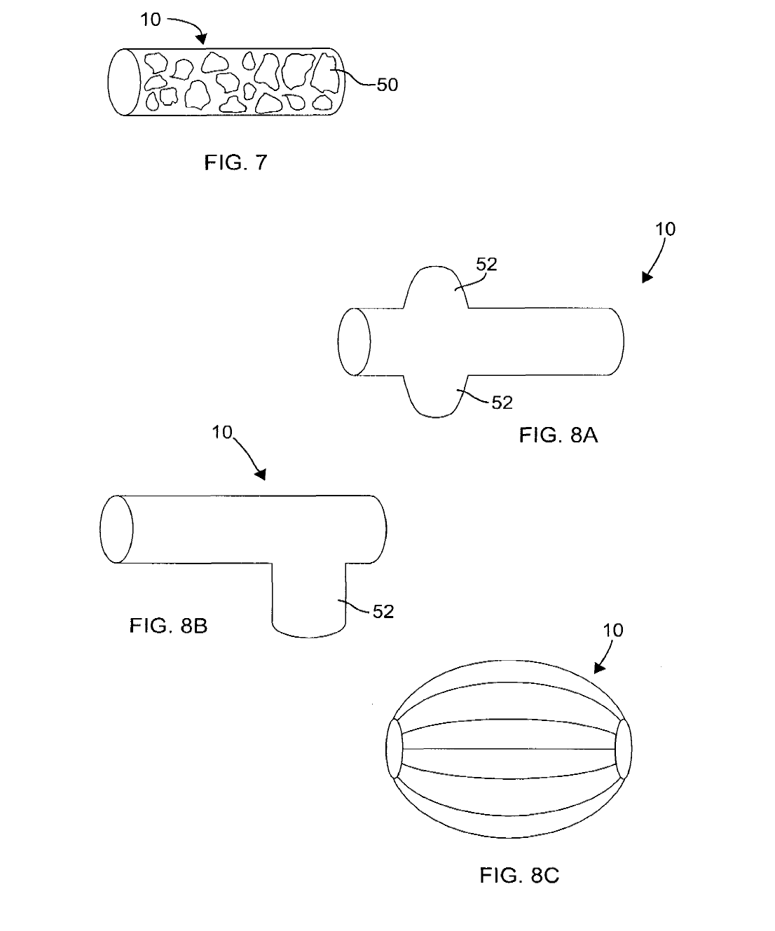

In alternative embodiments, stent 10 includes a plurality of cells 50, as shown in FIG. 7, and/or includes frame 12 having a high expansion ratio in selected areas or segments of stent 10, as shown in FIGS. 8A-8C. Each cell 50 may have substantially identical or similar dimensions as adjacent cells 50 or may have different dimensions. Cells 50 and/or the selected areas or segments of high expansion ratio facilitate fabricating stent 10 having selected areas or segments with different expansion ratios. As a result, stent 10 is fabricated in a suitable configuration having a suitable shape and/or suitable dimensions for an anticipated procedure. For example, stent 10 may have one or more areas or segments having an irregular shape with protrusions, invaginations and/or branches 52, as shown in FIGS. 8A and 8B, or stent 10 may have a substantially spherical shape, as shown in FIG. 8C. Additionally, or alternatively, stent 10 may include one or more openings or voids formed within the body wall of stent 10, in addition to the end openings. In a particular embodiment, such openings or voids are formed by positioning branches 52 at a selected angle with respect to the stent body wall and/or positioning branches 52 in a selected pattern, including an irregular pattern.

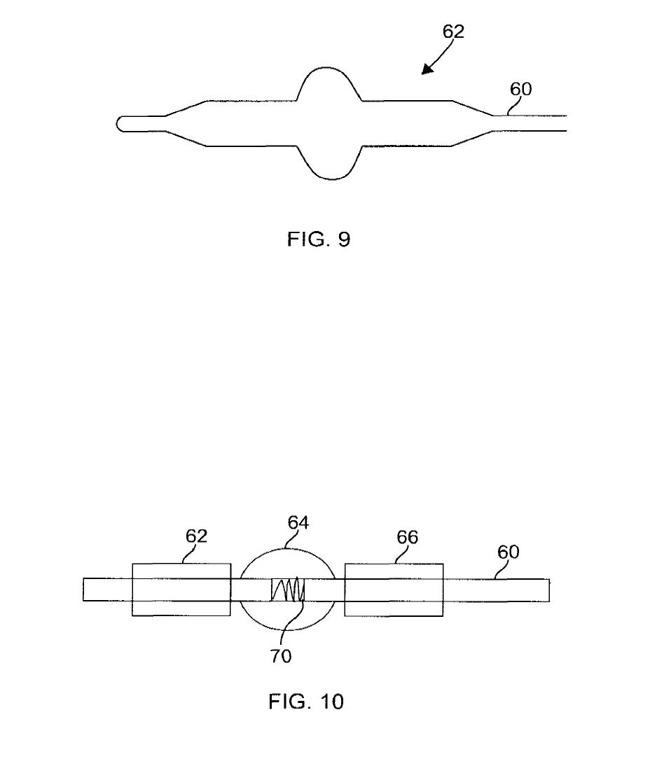

In one embodiment, a catheter 60 is utilized to facilitate properly positioning stent 10 within a body lumen. Catheter 60 includes at least one balloon 62 that is inflatable to expand stent 10 once properly positioned within the body lumen. Balloon 62 is configured to inflate to a desired shape, such as shown in FIG. 9. Alternatively, catheter 60 includes a series of balloons 62, 64, 66 having suitable configurations and/or sizes to create the desired shape, as shown in FIG. 10. Balloons 62, 64, 66 may have a common inflation channel or separate inflation channels (not shown). In a further embodiment, as shown in FIG. 10, one or more radio-opaque markers 70 including a radio opaque material are positioned on or with respect to catheter 60 to mark or identify a location of balloon 62, 64 and/or 66.

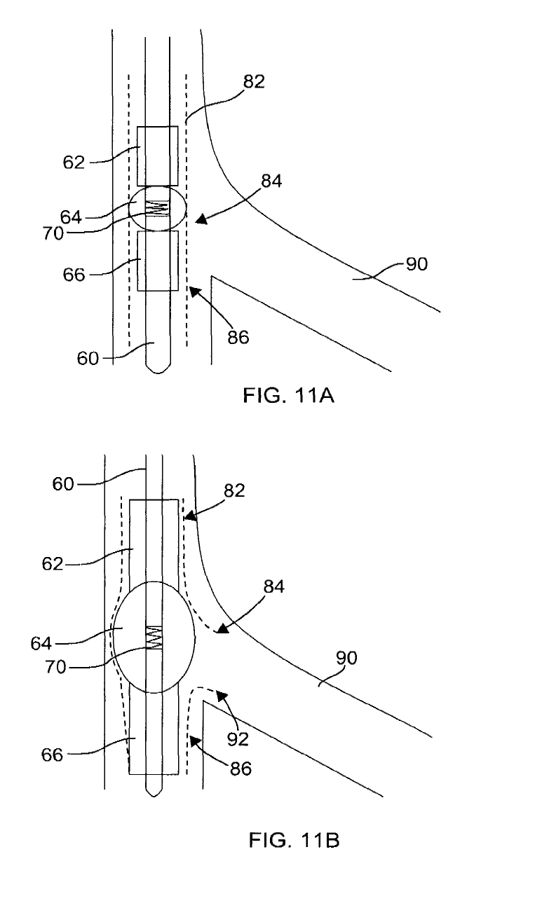

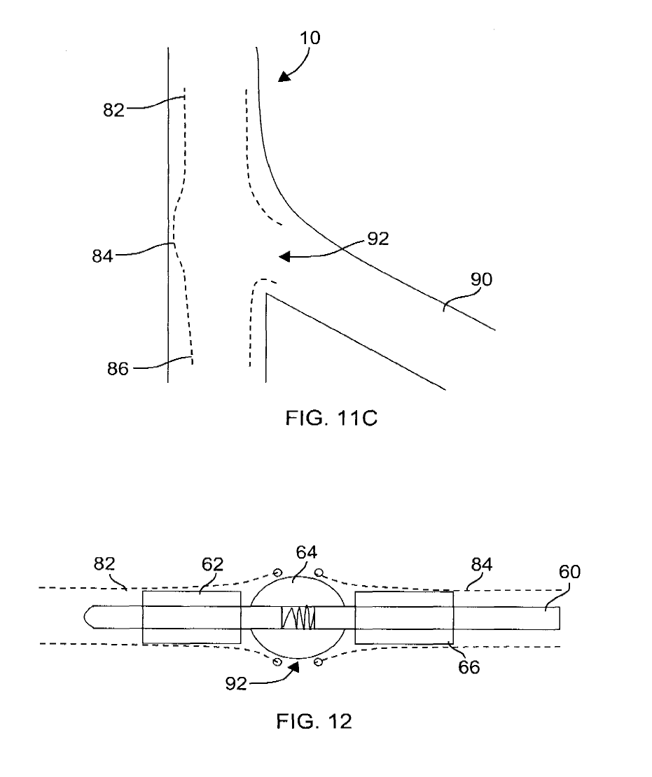

Referring to FIGS. 11A-11C, stent 10 is configured to treat a bifurcation within a body lumen. The stent 10 includes a plurality of cells or segments 82, 84, 86 each configured to expand radially outward or to open to define an opening. In a particular embodiment, one segment, such as segment 84, is configured to expand to a larger diameter than adjacent segments 82, 86 to form an opening within a body wall of segment 84. Segment 84 is positioned with respect to the bifurcation point and is configured to expand to the larger diameter to facilitate better flow into the branches, such as branch 90, and prevent or limit a compromise or closure of branch 90. In this embodiment, Catheter 60, with stent 10 coupled thereto, is inserted into the body lumen such that marker 70 is centered within the bifurcation point. With catheter 60 positioned such that marker 70 is visualized at the bifurcation point, balloons 62, 64, 66 are inflated to provide a suitable opening 92 in situ without the need to locate a correct side of the opening as needed with conventional stents having pre-made side openings. Moreover, the need to use guidewires or other conventional methods, such as rotating the conventional stent to locate the opening of side branch 90, in order to correctly position the stent side opening in a proper orientation with respect to branch 90, is obviated.

Referring further to FIG. 11B, balloons 62, 64, 66 are inflated to radially expand segments 82, 84, 86 of stent 10, respectively. As balloon 64 is inflated to radially expand segment 84, opening 92 is formed in a body wall of segment 84 to provide flow communication through stent 10 into branch 90. As shown in FIG. 11C, after stent 10 is properly positioned with respect to the bifurcation, catheter 60, including balloons 62, 64, 66, is removed from within the body lumen.

In a further alternative embodiment, stent 10 includes two substantially similar segments 82, 84. In a particular embodiment, segments 82, 84 are coupled together at a transition line. With catheter 60 properly positioned within the body lumen, balloons 62, 64, 66 are inflated to provide a suitable opening 92 at or near an area where segment 82 meets or transitions into segment 84, such as along the transition line. As balloon 64 is inflated to radially expand segment 82 and/or segment 84, opening 92 is formed to provide flow communication through opening 92. After stent 10 is properly positioned within the body lumen, catheter 60, including balloons 62, 64, 66, is removed from within the body lumen. Alternatively, the system may include two separate stents 10 positioned on catheter 60. The stents 10 may be coupled to each other or may be separate from each other.

The above-described device, such as a stent, has a high expansion ratio to facilitate accurately positioning the device in a collapsed or compressed configuration within a body lumen and to provide sufficient support to the body lumen upon expansion of the device to an expanded configuration within the body lumen. More specifically, the device includes an expandable frame that is movable between a collapsed, insertion configuration and an expanded, deployed configuration to facilitate providing a high expansion ratio for the device.

Exemplary embodiments of a device having a high expansion ratio are described above in detail. The device is not limited to the specific embodiments described herein, but rather, components of the device may be utilized independently and separately from other components described herein. Further, the described device can also be defined in, or used in combination with, other devices and/or methods, and are not limited to practice with only the device as described herein.

What is claimed is:

1. A device for expanding a stent to treat a bifurcation within a body lumen, the device comprising:
a catheter for delivering and positioning a stent to a bifurcation within a body lumen, the catheter having a balloon positioned adjacent to the stent, the balloon for being inflated to a predetermined configuration to expand the stent, the predetermined configuration having a first balloon portion, a central balloon portion, and a second balloon portion, the central balloon portion for being positioned adjacent to the bifurcation with the central balloon portion being inflated to expand the stent and to form an opening in the stent adjacent to the bifurcation to facilitate better flow into a branch associated with the bifurcation to prevent or limit a compromise or a closure of the branch, the central balloon portion being inflated to a larger diameter that the first balloon portion and the second balloon portion.

2. The device of claim 1 wherein the stent comprises at least one first member comprising a plurality of parallel bands spaced along the longitudinal axis, each band of said plurality of parallel bands comprising a plurality of substantially parallel first struts and a plurality of second struts, each second strut of said plurality of second struts coupled between adjacent first struts of said plurality of first struts and substantially perpendicular to said adjacent first struts with said frame in a collapsed configuration defining a plurality of first cells and a plurality of opposing second cells to facilitate expansion of said frame, said frame movable between the collapsed configuration and an expanded configuration, said frame comprising a first area having a first expansion ratio such that said first area is configured to selectively expand in a radial direction to a first expanded configuration having a first diameter, and a plurality of second areas spaced about a circumference of a body of said frame, each second area of said plurality of second areas having a second expansion ratio that is different from the first expansion ratio such that each second area of said plurality of second areas is configured to selectively expand in the radial direction to a second expanded configuration having a second diameter different than the first diameter and at least partially define an opening radially outward from said body of said frame when in the second expanded configuration, wherein the opening is orientated to provide flow communication into a branch extending from said body of said frame, wherein said frame does not include pre-made side openings; and
a plurality of second members coupled to a first band of said plurality of parallel bands, said plurality of first cells and said plurality of second cells positioned between adjacent second members of said plurality of second members, at least one second member of said plurality of second members comprising a plurality of third struts defining a plurality of third cells and a plurality of opposing fourth cells to facilitate expansion of said frame, wherein each third strut of said plurality of third struts is coupled to an adjacent third strut at an acute angle with said frame in the collapsed configuration, said at least one second member configured to expand in a longitudinal direction generally perpendicular to the radial direction to facilitate expansion of said frame along the longitudinal axis.

3. The device of claim 1 further comprising a marker positioned on the catheter to identify a location of the central balloon portion.

4. The device of claim 1 further comprising a first marker positioned on the catheter adjacent to the first balloon portion to identify a location of the first balloon portion, a second marker positioned on the catheter adjacent to the central balloon portion to identify a location of the central balloon portion, and a third marker positioned on the catheter adjacent to the second balloon portion to identify a location of the second balloon portion.

5. The device of claim 1 further comprising a marker positioned on the catheter to identify a location of the central balloon portion with the marker comprising a radio opaque material.

6. The device of claim 1 wherein the central balloon portion has a first diameter, the first balloon portion has a second diameter, and the second balloon portion has a third diameter with the first diameter being greater than the second diameter and the third diameter.

7. The device of claim 1 wherein the central balloon portion has a spherical shape when inflated.

8. The device of claim 1 wherein the central balloon portion has a spherical shape when inflated, the first balloon portion has a cylindrical shape when inflated, and the second balloon portion has a cylindrical shape when inflated.

9. The device of claim 1 wherein the central balloon portion has a spherical shape when inflated and the first balloon portion has a cylindrical shape when inflated.

10. The device of claim 1 wherein the central balloon portion has a spherical shape when inflated and the second balloon portion has a cylindrical shape when inflated,

11. A device for expanding a stent to treat a bifurcation within a body lumen, the device comprising:
a catheter for delivering and positioning a stent to a bifurcation within a body lumen, the catheter having a single inflation channel and a balloon positioned adjacent to the stent and in communication with the single inflation channel, the balloon for being inflated to a predetermined configuration to expand the stent by use of a fluid flowing through the single inflation channel, the predetermined configuration having a first balloon portion, a central balloon portion, and a second balloon portion, the central balloon portion for being positioned adjacent to the bifurcation with the central balloon portion being inflated to expand the stent and to form an opening in the stent adjacent to the bifurcation to facilitate better flow into a branch associated with the bifurcation to prevent or limit a compromise or a closure of the branch, the central balloon portion being inflated to a larger diameter that the first balloon portion and the second balloon portion.

12. The device of claim 11 wherein the central balloon portion has a first diameter, the first balloon portion has a second diameter, and the second balloon portion has a third diameter with the first diameter being greater than the second diameter and the third diameter when the first balloon portion, the central balloon portion, and the second balloon portion are inflated.

13. The device of claim 11 wherein the central balloon portion has a spherical shape when inflated.

14. A device for delivering and expanding a stent to treat a bifurcation within a body lumen comprising:
a stent comprising:
an expandable frame having a longitudinal axis, said frame comprising:
a first member comprising a plurality of substantially parallel bands spaced along a longitudinal axis, each band of said plurality of parallel bands comprising a plurality of substantially parallel first struts and a plurality of substantially parallel second struts with said frame in a collapsed configuration, each second strut of said plurality of second struts coupled between adjacent first struts of said plurality of first struts defining a plurality of first cells and a plurality of opposing second cells to facilitate expansion of said frame towards an expanded configuration, said frame comprising a first area having a first expansion ratio such that said first area is configured to selectively expand in a radial direction to a first expanded configuration having a first diameter, and a plurality of second areas spaced about a circumference of a body of said frame, each second area of said plurality of second areas having a second expansion ratio that is different from the first expansion ratio such that each second area of said plurality of second areas is configured to selectively expand in the radial direction to a second expanded configuration having a second diameter different from the first diameter and at least partially define an opening radially outward from said body of said frame when in the second expanded configuration, the opening oriented to provide flow communication into a branch extending from said body of said frame, wherein said frame does not include pre-made side openings;
a plurality of second members coupled to said first member, said plurality of first cells and said plurality of second cells positioned between adjacent second members of said plurality of second members, at least one second member of said plurality of second members comprising a plurality of third struts defining a plurality of third cells and a plurality of opposing fourth cells to facilitate expansion of said frame, wherein each third strut of said plurality of third struts is coupled to an adjacent third strut at an acute angle with said frame in the collapsed configuration, said at least one second member configured to expand in a longitudinal direction generally perpendicular to the radial direction to facilitate expansion of said frame along the longitudinal axis; and
a catheter for delivering and positioning the stent to a bifurcation within a body lumen, the catheter having a balloon positioned adjacent to the stent, the balloon for being inflated to a predetermined configuration to expand the stent, the predetermined configuration having a first balloon portion, a central balloon portion, and a second balloon portion, the central balloon portion for being positioned adjacent to the bifurcation with the central balloon portion being inflated to expand the cells of the stent adjacent to the central balloon portion to form an opening in the stent adjacent to the bifurcation to facilitate better flow into a branch associated with the bifurcation to prevent or limit a compromise or a closure of the branch, the central balloon portion being inflated to a larger diameter that the first balloon portion and the second balloon portion.

15. The device of claim 14 wherein the central balloon portion has a spherical shape when inflated.

16. The device of claim 14 wherein the central balloon portion has a spherical shape when inflated, the first balloon portion has a cylindrical shape when inflated, and the second balloon portion has a cylindrical shape when inflated.

17. The device of claim 14 wherein the central balloon portion has a spherical shape when inflated and the first balloon portion has a cylindrical shape when inflated.

18. The device of claim 14 wherein the central balloon portion has a spherical shape when inflated and the second balloon portion has a cylindrical shape when inflated.

19. The device of claim 14 wherein further comprising a marker positioned on the catheter to identify a location of the central balloon portion.

20. The device of claim 14 further comprising a first marker positioned on the catheter adjacent to the first balloon portion to identify a location of the first balloon portion, a second marker positioned on the catheter adjacent to the central balloon portion to identify a location of the central balloon portion, and a third marker positioned on the catheter adjacent to the second balloon portion to identify a location of the second balloon portion.
